(19) United States
(12) Patent Application Publication
(10) Pub. No.: US 2009/0298917 A1
(43) Pub. Date: Dec. 3, 2009

(54) Non-Viral Vector System For The Delivery Of Nucleic Acid Into The Lung

(76) Inventors: Thomas Kissel, Staufen (DE); Elke Kleemann, Mainz (DE); Michael Neu, Edingen-Neckarhausen (DE); Tobias Gessler, Wettenberg (DE); Thomas Schmehl, Giessen (DE)

Correspondence Address: CLARK & BRODY, 1090 VERMONT AVENUE, NW, SUITE 250, WASHINGTON, DC, 20005, US

(21) Appl. No.: 11/920,500

(22) PCT Filed: May 20, 2006
(86) PCT No.: PCT/DE2006/000875

§ 371 Date: Apr. 21, 2009

(30) Foreign Application Priority Data
May 20, 2005 (DE) 10 2005 023 993.5

(51) Int. Cl. A61K 31/7088; C12N 15/63; A61P 11/00; A61P 35/00

(52) U.S. Cl. 514/44 R; 435/320.1

(57) ABSTRACT

The present invention related to a non-viral vector system for the delivery of nucleic acids which is modified on the basis of polyethylene imine (PEI) with polyethylene glycol (PEG) and which contains a peptide sequence with PTD/CPP-functionality.

This vector system is used as gene transfer system for the epithelial cells of the bronchial tubes and alveoli of the lung. The system is highly stable, protects the DNA in the pulmonary environment, has a low zeta-potential and a low aggregation tendency in a medium with a high ionic strength. Furthermore, it is characterized by a low in vitro and in vivo cytotoxicity and allows for a very high transfection efficiency. The vector system according to the present invention is used for a transfection by inhalation in local pulmonary therapy.

The present invention concerns a non-viral vector system for the delivery of nucleic acids which is based upon polyethylene imine (PEI) and modified with polyethylene glycol (PEG) and which has a peptide sequence with PTD/CPP-functionality. This system is, due to a low toxicity and high stability, especially suited for the delivery of nucleic acids to the lung and thus for the treatment of pulmonary diseases.

### BACKGROUND OF THE PRESENT INVENTION

One of the main problems in gene therapy and the treatment of diseases with genetic material associated therewith is the discovery of suitable means of transport in order to deliver the genetic material to the respective target cells where this material can be expressed.

The lung is an important organ at the interface between environment and internal body parts. Gaseous substances or particles may reach the various branches of the lungs via the respiratory tract which is directly connected with the environment, and are able to interact here with the inner surface border of the respiratory system. Particularly for the treatment of pulmonary diseases such as pulmonary hypertony, mucoviscidosis, and lung tumors, there is a high demand for vehicles for DNA.

In order to be applicable in vivo, a vector system generally has to meet the following requirements:
tissue specificity:
   the vector system has to be cell-target specific, especially when the vector system is supposed to transport the genes to be expressed only into a specific cell type
size of DNA transferred
   the vector system should tolerate a use of DNA of unlimited size and also be able to deliver large genes
immunogenicity
   the vector system itself should not be immunogenic, i.e. it should not elicit an immune answer in the transfected cell or in the patient
toxicity
   the vector system itself should not display any cytotoxic effects on the cells
stability
   the vector system should be stable with respect to degradation by endo- and exonucleases and should also, depending on the application range, be stable with respect to the form of application, e.g. spray application
size
   the vector system should be as small as possible in order to be transferred into the cell or to or into the cell nucleus
cell penetration and nuclear transport
   the vector system should be transported into the cell, into or to the nucleus with high transfection efficiency
producibility
   for commercial application, production, storage, and transport of the vector system should be easy and cost efficient.

The state of the art knows vector systems based on viral vectors such as e.g. retroviral, adenoviral or adenoviral-associated vector systems, as well as non-viral vectors such as e.g. polyethylene imines or dendrimeres and the application of naked DNA using physical methods such as e.g. electroporation or the gene gun. In principle, viral vectors used as transfection and expression vectors are highly efficient for the delivery of genes into mammalian cells, but the uptake capacity of these cells for therapeutic foreign DNA is very limited and they always display immunogenic features to a varying degree. This means that even though genes are transferred in a more or less target-specific manner, cells may also be damaged and a strong reaction of the immune system may be elicited in this process.

In order to avoid problems which are caused by the side effects of viruses, scientists have developed non-viral transport systems. Non-viral vectors use naked DNA which codes for a specific protein and is supplied with the regulatory ele-ments required for the expression of this protein sequence. This naked DNA is transferred into the cell using various means, is not integrated stably into the genome but remains extra-chromosomally, leading to an endogenous synthesis of the protein. Alternatively, the genes to be transferred are coated in liposomes or polymers and can be transferred safely into the cells via membrane fusion and endocytosis, respectively. So-called lipoplexes are already used to date for the therapy in humans, e.g. for the treatment of mucoviscidosis and black skin cancer (malignant melanomas). The use of naked DNA as vector system is of advantage since this DNA can easily be manipulated and thus be used for gene therapy.

Plasmids are small extra-chromosomal DNA-fragments which are especially suitable as vectors to deliver large genes into a broad variety of cells. Plasmids are routinely used for the gene transfer from one to another organism. In contrast to viral vectors, plasmids can be prepared cost-efficiently, in high amounts, and with good quality.

It is the aim of the present invention to provide a non-viral vector system which is suitable for the delivery of nucleic acids, being characterized in that this system has a high transfection efficiency and low toxicity and is suitable for the application in the lung, as well as an easily practicable and cost efficient production procedure.

The problem is solved by the present invention according to claim 1 in that a vector system is provided for the delivery of nucleic acids with polyplexes consisting of polyethylene imine (PEI), polyethylene glycol (PEG), and a peptide with PTD/CPP functionality, as well as a procedure for the production thereof according to claim 18.

Especially advantageous features of the vector system according to the present invention consisting of a peptide with PTD/CPP functionality, PEG, and PEI are:
enhanced stability and protection of the enclosed nucleic acid, especially in the pulmonary environment
the zeta-potential and thus the surface-charge of the polyplexes is reduced, leading to a low cytotoxicity in vitro and in vivo, for example in lung epithelial cells
polyplexes are characterized by a reduced aggregation tendency in media with high ionic strength
a 600% increase of transfection efficiency in vivo, as compared to polyethylene imine 25 kDa (PEI)
transport of nucleic acid such as e.g. DNA, RNA, and/or plasmid-DNA directly into the epithelial cells of bronchial tubes and alveoli
application in the local pulmonary therapy

Surprisingly it was found that vector systems on the basis of PEI show a particularly good stability and high transfection efficiency under in vivo conditions when these systems are linked to peptide sequences with PTD/CCP functionality.

According to the present invention, a peptide with PTD/CCP functionality is a peptide with a protein transduction domain (PTD) or a cell penetration domain (CPP). Preferably, this is the TAT-peptide or a peptide sequence related to the TAT-peptide. The TAT-peptide is a membrane peptide of 86 amino acids which derives from the HIV-virus (human immune deficiency virus type 1).

Further peptides with a protein transduction domain or a cell penetration domain are known to the experts, among these MAP, Antp, and MAT, as well as derivatives thereof. As linker, preferably polyethylene glycol (PEG) is used, but other suitable linkers can be used as well.

Transfection generally means the introduction of a nucleic acid, preferably foreign DNA into cells, whereby the introduction of the nucleic acid can be transient or stable.

The nucleic acid codes preferably for substances which are, alone or in combination, supposed to cause a positive effect in the treatment of pulmonary diseases. In this context, the nucleic acid can be DNA or RNA.

The synthesis of the polymer conjugate is performed by modifying the C-terminus of an oligopeptide with PTD/CPP functionality for example at a cystein-residue and covalent coupling to PEI via the hetero-bifunctional linker, for example PEG.

The non-viral vector system for the delivery of nucleic acids according to the present invention shows outstanding features for the use in the lung in order to treat patients who suffer from pulmonary diseases.

In this context, the term “patient” denotes likewise human beings and vertebrates. The non-viral vector system for the delivery of nucleic acids according to the present invention can thus be used in human and veterinary medicine. Preferably, this system is used for the production of remedies for the treatment of pulmonary diseases such as pulmonary hypertony, mucoviscidosis, and lung tumors.

The non-viral vector system for the delivery of nucleic acids according to the present invention is applied to the patient, as part of a pharmaceutically acceptable composition, either by inhalation, orally, rectally, parental intravenously, intramuscularly or subcutaneously, intra-cisternally, intra-vaginally, intra-peritoneally, intra-vascularly, locally (powder, ointment, or drops), via intra-tracheal intubation, intra-tracheal instillation, or as spray.

Pharmaceutically acceptable compositions include modifications as salts, esters, amides, and prodrugs, as far as these compositions do not elicit increased toxicity, irritations, or allergic reactions in the patient according to a reliable medical evaluation.

The terminus “prodrug” denotes compounds with are transformed in order to improve the uptake, such as for example by hydrolysis in the blood.

Pharmaceutical forms for a local administration of this invention include ointments, powders, sprays, or inhalants. The active compound is mixed under sterile conditions with a physiologically acceptable carrier and possible preservatives, buffers, or propellants, if required.

### DETAILED DESCRIPTION OF THE INVENTION

The non-viral vector system for the delivery of nucleic acids is a polymer conjugate consisting of PEG, PEI, and peptide sequences with PTD/CPP-functionality. Exemplarily for a protein with PTD/CPP-functionality is the TAT-peptide or a peptide sequence which is related to the TAT-peptide.

Exemplarily for a sequence which is related to the TAT-peptide is the decapeptide sequence GRKKKRRQRC. Further TAT-peptide related sequences are well-known and can be used alternatively. These are synthesized by order e.g. by the company Bachem.

Other materials are:

Plasmid pGL3 harboring a luciferase-encoding region under the promoter control of the cytomegalovirus (CMV), can be purchased from Promega GmbH and is propagated in E. coli, isolated, and purified according to the standard protocol as recommended by the manufacturer.

Herring testes DNA is commercially available e.g. from the company Sigma.

Plasmid peGFP-N1 harboring the green fluorescence coding region under the control of the cytomegalovirus (CMV-N1) promoter can be purchased from Clon-Tech (1290 Terra Bella Avenue Mountain View, Calif. 94043 USA) and is propagated in E. coli, isolated, and purified according to the standard protocol as recommended by the manufacturer. The natural surfactant Alveofact® is commercially available from e.g. Boehringer-Ingelheim. Bronchial alveolar lavage fluid (BALF) is obtained according to a standard procedure from C57BL/6 mice via intra-tracheal instillation. For a removal of interfering cells, BALF is centrifuged e.g. at 300 g and 4° C. and the supernatant is used for further treatment.

The procedure for the production of a non-viral vector system for the delivery of nucleic acids includes the synthesis of polymer conjugate composed of PEG and PEI and the coupling to peptide sequences with PTD/CPP-functionality such as for example the TAT-peptide or a TAT-like peptide.

TAT-PEG-PEI polyplexes containing PEI, e.g. a 25 kDa branched PEI, a PEG-linker, and a TAT-like oligopeptide sequence as for example GRKKKRRQRC are synthesized according to the following reaction scheme:
i) Reaction of bifunctional branched polyethylene glycol (PEG) containing an α-vinyl sulfone and an ω-N-hydroxysuccinimide ester group (NHS-PEG-VS) with polyethylene imine (PEI) to active PEI (VS-PEG-PEI)
ii) Reaction of the activated PEI (VS-PEG-PEI) with a peptide with PTD/CPP-functionality
iii) Separation of polymer complexes from non-bound PEG and low-molecular weight residues

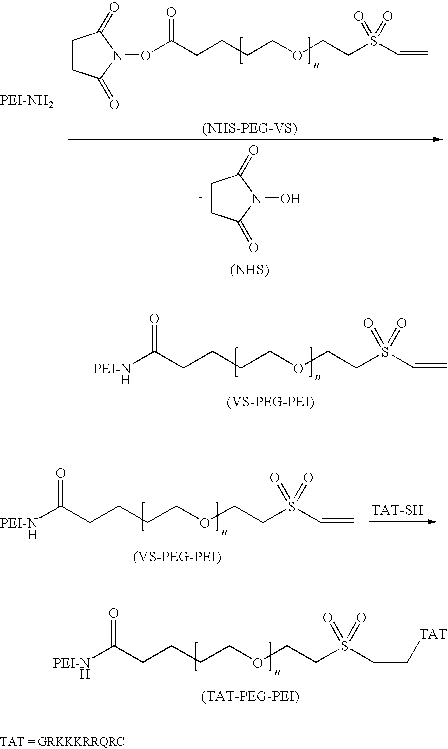

Starting substance for the procedure is branched bifunctional polyethylene glycol (PEG) containing an α-vinyl sulfone- and an ω-N-hydroxysuccinimide ester group (NHS-PEG-VS), as for example commercially available from the company Nektar Therapeutics (Huntsville, USA).

NHS-PEG-VS is reacted at a pH-value of 5.5 with polyethylene imine (PEI), which can be obtained e.g. from BASF.

PEI activated in this manner (VS-PEG-PEI) is coupled in a further reaction with the TAT-like peptide by a reaction via SH-group.

### Activation of PEI

19.2 mg (565 μmol) bifunctional PEG (3.4 kDa) containing an α-vinyl sulfone- and an ω-N-hydroxysuccinimide-group are given into a flask. 4.293 ml of a PEI solution (corresponding to 12.15 mg/0.486 μmol PEI; 282.4 μmol total amine in 0.1M borate buffer pH 5.5) are added and the mixture is stirred. The activation reaction is continued for 4 hours at room temperature. The pH-value is adjusted to pH 7 with 1 N hydrochloric acid and the reaction is continuously stirred for 2 hours at room temperature.

### Coupling of the Oligonucleotide to Activated PEG-PEI

For the coupling reaction, 2.98 mg (2.26 μmol) of the decapeptide GRKKKRRQRC are dissolved in 866 μl water. The peptide solution is added to the activated PEG-PEI and the reaction is continued for 2 hours at room temperature.

### Purification of TAT-PEG-PEI

Non-bound PEG or peptide and low molecular weight residues are removed, e.g. by using an ultrafiltration cell (Amicon, Bedford, USA) equipped with a 10 kDa molecular weight cut-off membrane (Millipore, Bedford, USA) and 0.1M borate buffer at pH 7.5 as eluent.

### Complex Formation

For the complex formation, conjugate or DNA, respectively, are separately diluted to the desired concentration with a 5% glucose solution at pH 7.4. Subsequently, the polymer solution is rapidly added to the nucleic acids, for example the DNA, and mixed thoroughly by vigorous pipetting up and down, followed by an incubation time for 10-20 minutes at room temperature.

### Measurement of DNA Condensation Ability in an Ethidium Bromide Exclusion Assay

The quenching of ethidium bromide (EtBr)-fluorescence is a measure for the DNA condensation ability.

8 μg herring or salmon testes DNA in 60 mM Tris buffer pH 7.4 are each complexed in 96-well plates with increasing amounts of PEI or TAT-PEG-PEI in a total volume of 280 μL buffer. After 10 minutes incubation time, 20 μl EtBr-solution (0.1 mg/ml) are added. The fluorescence is measured at λₑₓ=518 nm and λₑₘ=605 nm e.g. on a Perkin Elmer LS 50 B fluorescence plate reader.

Free ethidium bromide is indicated by a weak fluorescence. This fluorescence increases when ethidium bromide intercalates into DNA. FIG. 1 shows the ethidium bromide exclusion assay. The quenching of DNA/ethidium bromide fluorescence is indicated here as ₙrelative fluorescence″, whereby 100% corresponds to the control consisting of DNA with ethidium bromide without polymer. Displayed is the quenching for PEI (black circle) and TAT-PEG-PEI (black triangle) in the presence of salmon testes DNA. Values are given as mean values±SD (n=4). It becomes apparent that TAT-PEG-PEI shows a stronger quenching of fluorescence and thus a higher DNA condensation ability than pure PEI 25 kDa.

### Physical-Chemical Properties

Important parameters for a more efficient gene transfer into cells are the zeta-potential and the particle size of polyplexes according to the present invention.

The endocytotic uptake of particles increases with increasing zeta-potential, but at the same time toxic side effects due to unspecific interactions between the particles and the cell membrane are enhanced.

For the gene transfer into cells it is thus desirable to possess particles with highly positive zeta-potential and simultaneously low cytotoxicity.

In addition, the gene transfer into lung cells is characterized by further complications, e.g. a mucus layer which inhibits the cell contact of polyplexes, leading to a removal of particles from the lung (mucociliary clearance). Furthermore, phagocytosis by alveolar macrophages is another source of loss of polyplexes. The endocytotic uptake of particles is generally dependent on the particle size which consequently increases with a reduction of particle size. Thus it is desirable to have particles for the gene transfer into lung cells which are small and also stable at various N/P ratios in different media.

The expert skilled in the art knows that the N/P ratio indicates the ratio of DNA to PEI, given as molar ratio of nitrogen atoms of PEI to phosphor atoms of the DNA.

The TAT-PEG-PEI-polyplexes according to the present invention meet these demands as a non-viral vector system for the delivery of nucleic acids.

The measurement of the surface charge and the corresponding zeta-potential is performed using laser Doppler anemometry. While naked DNA shows a strongly negative zeta-potential of −33 mV, complexes according to the present invention exhibit a positive zeta-potential. For TAT-PEG-PEI-polyplexes, a zeta-potential of 15±3 mV to 20±3 mV is observed (dependent on the N/P-ratio), PEI 25 kDa-complexes have a zeta-potential of 32 mV.

FIG. 2 demonstrates the results of the particle size measurement of TAT-PEG-PEI-polyplexes and plasmid-DNA in comparison.

Part A shows the hydrodynamic diameters TAT-PEG-PEI/pGL3-polyplexes in various media. The dark bar refers to results obtained in NaCl 150 mM pH 7.5, the light bar to results obtained in 5% glucose 5% pH 7.5.

Part B shows a comparison of TAT-PEG-PEI-polyplexes to PEI-polyplexes with respect to their aggregation tendency in media with high ionic strength (NaCl 150 mM at pH 7.5). Compared are PEI 25 kDa-polyplexes which were synthesized at an N/P-ratio of 3 (black squares), PEI 25 kDa polyplexes at an N/P-ratio of 7 (black triangles), TAT-PEG-PEI-polyplexes at an N/P-ratio of 3 (white squares), and TAT-PEG-PEI-polyplexes at an N/P-ratio of 7 (white triangles).

Polyplexes according to the present invention have a size of 135 to 176 nm in 150 mM NaCl-solutions, in 5% glucose-solutions a size of 89 to 107 nm at pH 7.5. A smaller particle size is observed for TAT-PEG-PEI/DNA in glucose-solution when the N/P-ratio increases from 3 to 10. Polyplexes synthesized with an N/P ratio of 3 show a larger particle size.

TAT-PEG-PEI-polyplexes produced in media with high ionic strength (150 mM NaCl) are stable over a period of 20 minutes, while PEI polyplexes as control show a drastic increase of the particle size due to an aggregation.

### Stability of Polyplexes

TAT-PEG-PEI polyplexes with and without DNA are investigated with respect to complex stability regarding polyanions and enzymatic degradation in the environment of the lungs. The stability of polyplexes is demonstrated experimentally in the presence of heparin, Alveofact®, BALF, and DNase I (FIG. 3-5).

For this, polyplexes (N/P ratio of 8) are incubated with increasing amounts of heparin (FIG. 3). PEI protects plasmid-DNA against heparin exchange up to 0.2 IU heparin. An amount of 0.5 IU heparin results in a release of the DNA from the PEI-polyplexes. TAT-PEG-PEI-polyplexes are stable even in the presence of 0.5 IU heparin and do not release DNA.

FIG. 3 demonstrates the results of these experiments concerning the stability of polyplexes in the presence of heparin. PEI and TAT-PEG-PEI-polyplexes (N/P ratio of 8) are incubated with increasing amounts of heparin (0.1, 0.2, 0.5, 1, 1.5, 2, IU heparin per 1 μg pGL3).

The different DNA-morphologies are labeled with A: supercoiled, B: linear, and C: open circular. D indicates the start line.

Stability assays with Alveofact® and BALF are performed using the reverse EtBr exclusion assay and are shown in FIG. 4. In the absence of Alveofact® and BALF, the ability for DNA condensation was evident up to a fluorescence of 8% (PEI) and 5% (TAT-PEG-PEI). The addition of Alveofact® increased the fluorescence for both polyplexes in a concentration dependent manner. At an Alveofact®-concentration of 2 μg/μl, PEI/DNA exhibits a stability of 14.1% and TAT-PEG-PEI-polyplexes a stability of 11.8%. The fluorescence increasing with DNA release of PEI-polyplexes in BALF increases in a linear manner from 8% (0 min) up to 18.5% relative fluorescence (90 min), while the fluorescence of TAT-PEG-PEI-polyplexes (1,4%) did not increase over the observation period of 90 min. This demonstrates that DNA in TAT-PEG-PEI polyplexes is protected against extracellular pulmonary enzymes and proteolipids.

FIG. 4 shows the results after treatment of PEI and TAT-PEG-PEI-polyplexes with an N/P-ratio of 8 with increasing amounts of Alveofact® (gray line) and BALF (black line).

FIG. 5 shows the results of the DNA digestion assay with nuclease DNase 1. TAT-PEG-PEI-polyplexes are incubated for 15 minutes with increasing concentrations of DNase I. DNA digest occurres at a concentration 2.5 IU DNase I per 1 μg DNA. Only above 5 IU, a complete degradation of plasmid DNA is observed.

In addition to the stability with respect to intracellular enzymes (e.g. in endosomes, lysosomes) the non-viral vector system for the delivery of nucleic acid according to the present invention is also very stable in an extracellular environment and thus suitable for an efficient use in the lung. As compared to PEI, the stability of TAT-PEG-PEI-polyplexes is significantly higher in the presence of high concentrations of heparin, Alveofact®, BALF, and DNase I.

### Transfection Efficiency

In order to investigate the transfection efficiency, plasmid DNA is complexed with PEI or TAT-PEG-PEI (N/P-ratios of 8 and 10). FIG. 6A indicates the in vitro results and FIG. 6B the in vivo results in mouse lungs.

While the transfection efficiency of TAT-PEG-PEI-polyplexes in lung epithelial cells A549 is lower as compared to the efficiency of PEI 25 kDa-polyplexes, a high gene expression results in experiments with mouse lungs. At an N/P-ratio of 10, a high gene expression was observed for TAT-PEG-PEI (12.6 pg luciferase/mg lung tissue) and a lower expression for PEI (2 pg luciferase/mg lung tissue).

Taken together, TAT-PEG-PEI-polyplexes show a 600% increase of transfection efficiency in vivo as compared to PEI.

### Toxicity

The metabolic and mitochondrial activity of cells is determined with the colorimetric MTT-assay. FIG. 7A shows the toxicity of polymers depending on their concentration. PEI reduces the viability of cells at concentrations of 0.1 mg/ml (˜23% viability), TAT-PEG-PEI-polyplexes exhibit only little influence on the cells (˜70% viability). IC₅₀ values are in the following range: for BPEI 0.071 mg/ml, for TAT-PEG-PEI 0.2 mg/ml.

FIG. 7B indicates cell counts of PEI-polyplexes after treatment in the lung, FIG. 7C shows the total protein concentration in BALF, and FIG. 7D shows the experiments to increase the TNF-α levels.

### Distribution of Polyplexes in the Mouse Lung

To evaluate the distribution of polyplexes, double-labeled TAT-PEG-PEI/plasmid-polyplex is applied to the mouse lung and the distribution is determined in formaldehyde-fixed cryosections four hours post application.

Plasmid-DNA and polymer are largely co-localized. The double-labeled polyplexes are localized in the bronchial epithelial cells and the alveolar region. These results confirm the high applicability of TAT-PEG-PEI-polyplexes as a vector system for various diseases of the lung.

### LIST OF FIGURES

FIG. 1 shows the fluorescence quenching of ethidium bromide (relative fluorescence in %) in an assay. Shown is the quenching for PEI (black circle) and TAT-PEG-PEI (black triangle) in the presence of salmon testes DNA. Values are given as mean values±SD (n=4).

FIG. 2 shows results with respect to particle size

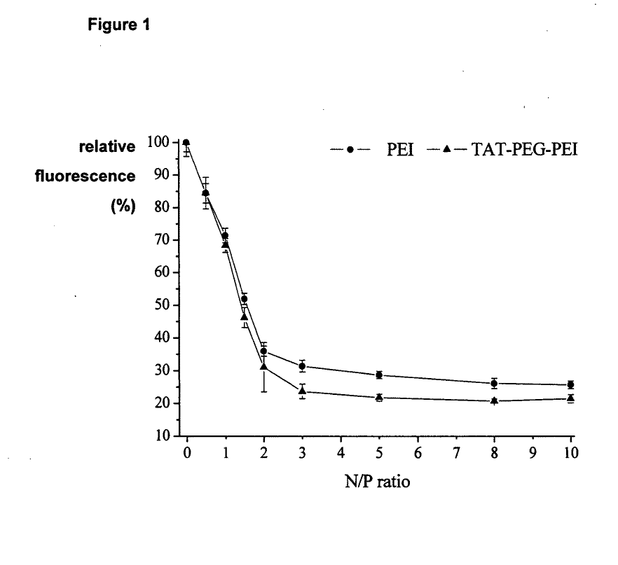

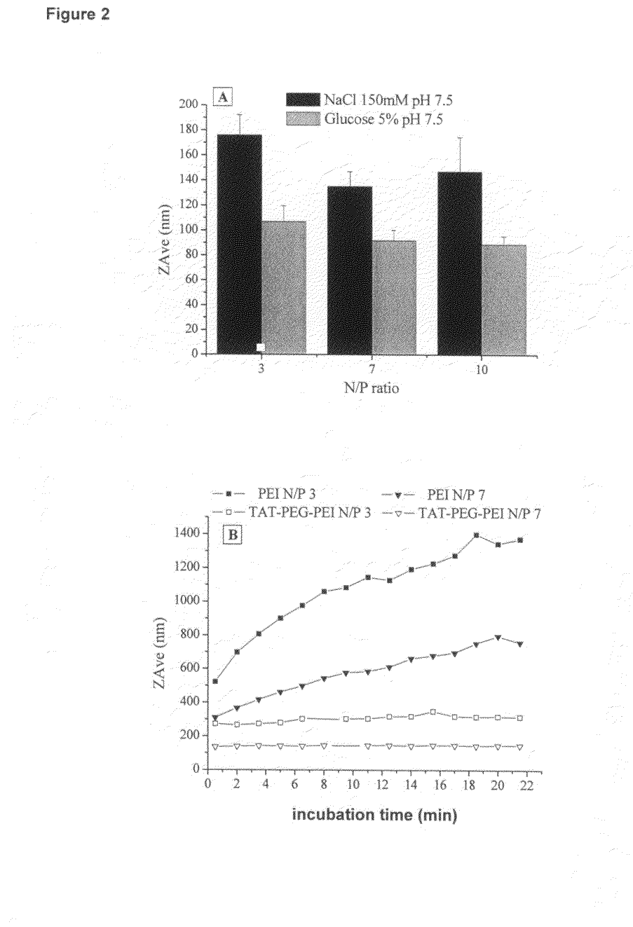

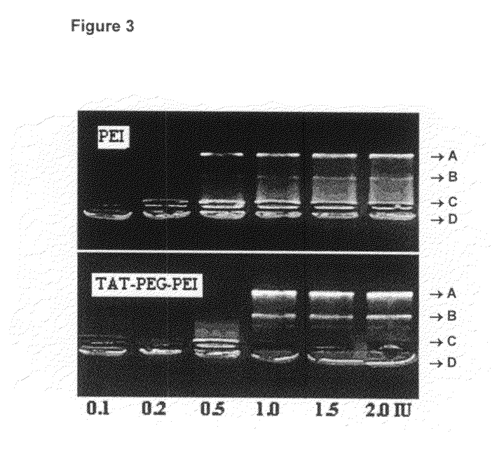

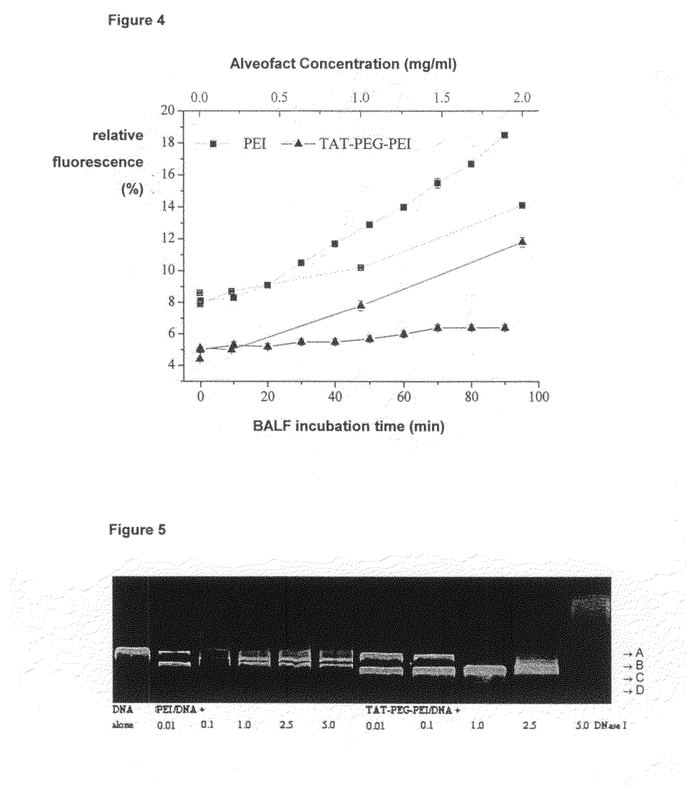

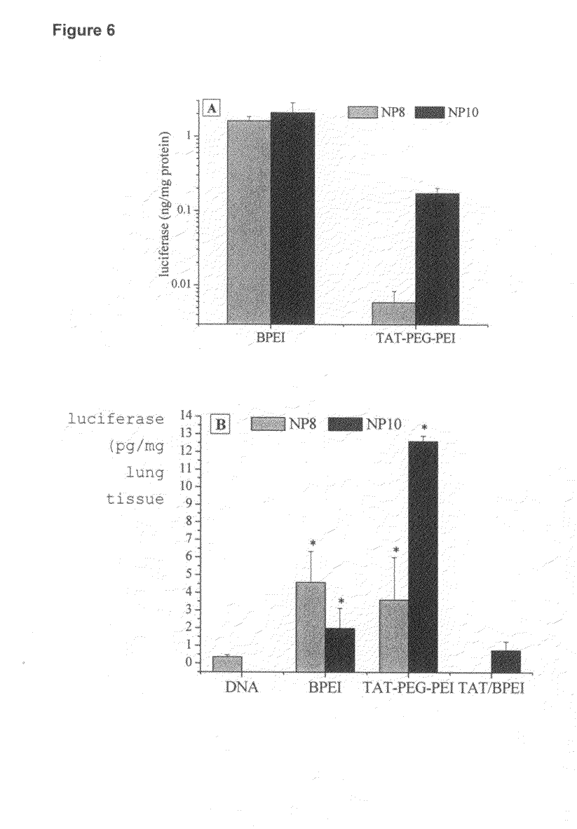

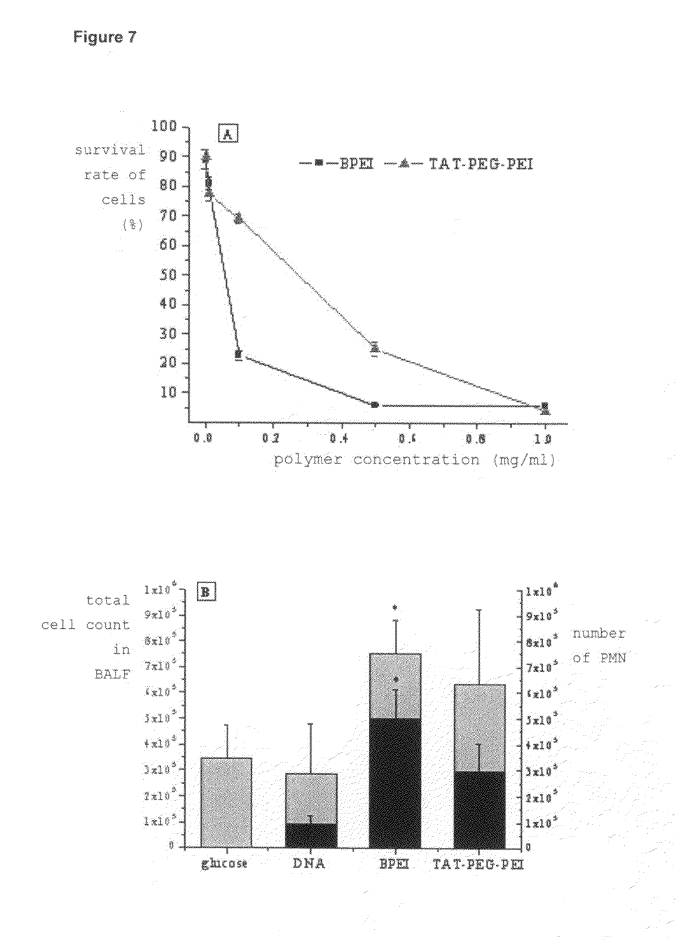

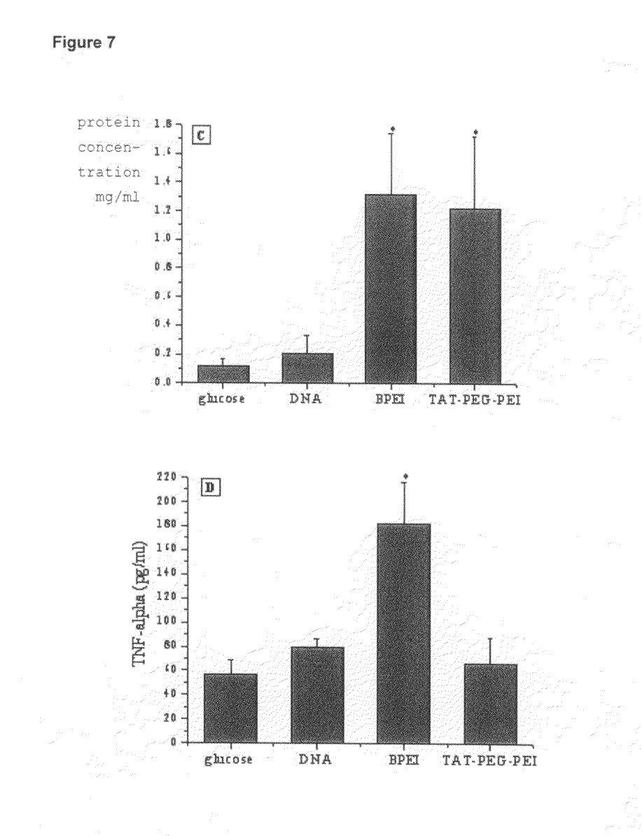

Part A demonstrates the hydrodynamic diameters of TAT-PEG-PEI/pGL3-polyplexes in various media. The dark bar refers to results obtained in NaCl 150 mM pH 7.5, the lightly colored bar refers to results in glucose 5% pH 7.5.

Part B demonstrates a comparison of TAT-PEG-PEI-polyplexes to PEI-polyplexes with respect to their aggregation tendency in medium with high ionic strength (NaCl 150 mM at pH 7.5). Compared are PEI-polyplexes with an N/P ratio of 3 (black squares), PEI-polyplexes with an N/P-ratio of 7 (black triangles), TAT-PEG-PEI-polyplexes with an N/P-ratio of 3 (white squares), and TAT-PEG-PEI-polyplexes with an N/P-ratio of 7 (white triangles).

FIG. 3 shows the stability of polyplexes in heparin.

PEI and TAT-PEG-PEI-polyplexes (N/P-ratio of 8) are incubated with increasing amounts of heparin (0.1, 0.2, 0.5, 1, 1.5, 2, IU heparin per 1 μg pGL3).

The different DNA-morphologies are labeled with A: supercoiled, B: linear, and C: open circular. D indicates the start line.

FIG. 4 shows the results of a treatment of PEI and TAT-PEG-PEI-polyplexes (N/P ratio of 8) with increasing amounts of Alveofact® (gray line) and BALF (black line). Values are indicated with standard deviation ±SD and depicted as percentage of maximum fluorescence (pGL3 alone).

FIG. 5 shows PEI and TAT-PEG-PEI-polyplexes (N/P-ratio of 8) and plasmid-DNA with increasing amounts of DNase I (upper picture: PEI 25 kDa, lower picture: TAT-PEG-PEI-polyplexes) (0.01, 0.1, 1, 2.5, and 5 IU DNase I per 1 μg pGL3) Lane 1 shows free undigested DNA pGL3-control. The different DNA-morphologies are labeled with A: supercoiled, B: linear und C: open circular. D indicates the start line.

FIG. 6 shows the transfection efficiency of polyplexes at N/P-ratios of 8 and 10: A) luciferase-expression in A549, B) luciferase-expression in C57BU6 mouse lung. The in vivo results are given ±SD.

FIG. 7 shows the results of toxicity studies: A) Cytotoxic effects of the polymers at concentrations of 0.001, 0.01, 0.1, 0.5, and 1.0 mg/ml on A549 cells, determined by MTT-assay and presented as relative cell viability (mean±SD of 6 determinations). Pulmonary inflammation indicators (mean values±SD of 3 determinations) in the BALF 24 post application of polyplexes (N/P ratio of 10) in mouse lungs: B) total cell counts and number of PMN, C) total protein concentration, D) TNF-alpha-concentration.

1. Non-viral vector system for the delivery of nucleic acids, which is characterized in that it includes polyplexes of polyethylene imine (PEI), polyethylene glycol (PEG), and a peptide sequence with PTD/CPP-functionality.

2. Non-viral vector system according to claim 1 which is characterized in that the peptide sequence with PTD/CPP-functionality comprises the TAT peptide.

3. Non-viral vector system according to claim 1 which is characterized in that the peptide sequence with PTD/CPP-functionality comprises a TAT-like peptide.

4. Non-viral vector system according to claim 3 which is characterized in that the peptide sequence with PTD/CPP-functionality comprises the decapeptide GRKKKRRQRC.

5. Non-viral vector system according to claim 1 which is characterized in that the nucleic acid is RNA and/or DNA.

6. Non-viral vector system according to claim 1 which is characterized in that has a zeta-potential of 15±3 mV to 20±3 mV.

7. Non-viral vector system according to claim 1 which is characterized in that it exhibits a reduced aggregation tendency in a medium with high ionic strength.

8. Non-viral vector system according to claim 1 which is characterized in that it is stable in a medium with high ionic strength.

9. Non-viral vector system according to claim 7 which is characterized in that it is stable for more than 20 minutes in a medium with high ionic strength.

10. Non-viral vector system according to claim 7 which is characterized in that the medium with high ionic strength is 150 mM NaCl.

11. Non-viral vector system according to claim 1 which is characterized in that this system exhibits low in vivo cytotoxicity in pulmonary epithelial cells.

12. Non-viral vector system according to claim 1 which is characterized in that this system exhibits low in vivo cytotoxicity in pulmonary epithelial cells.

13. Non-viral vector system according to the claim 1 which is characterized in that the nucleic acids are directly transported into the epithelial cells of bronchial tubes and alveoli.

14. Non-viral vector system according to claim 1 which is characterized in that the transfection is performed by inhalation.

15. Non-viral vector system according to claim 1 which is characterized the transfection is performed by intra-tracheal instillation.

16. Non-viral vector system according to claim 1 which is characterized in that it is utilized as remedy for the treatment of pulmonary diseases.

17. Non-viral vector system for the delivery of nucleic acids, which is characterized in that it includes polyplexes of polyethylene imine (PEI), polyethylene glycol (PEG), and a peptide sequence with PTD/CPP-functionality and produced according to a procedure according to claim 18.

18. Procedure for the production of a vector system for the delivery of nucleic acids into the lung, characterized by the steps of:
i) Reaction of branched bifunctional polyethylene glycol (PEG) containing an α-vinyl sulfone- and an ω-N-hydroxysuccinimide ester group (NHS-PEG-VS) with polyethylene imine (PEI) to give activated PEI (VS-PEG-PEI)
ii) Reaction of the activated PEI (VS-PEG-PEI) with a peptide with PTD/CPP-functionality
iii) Separation of the polymer complexes from non-bound PEG and low molecular weight residues.

19. Procedure according to claim 18 which is characterized in that step i) is performed at pH 5.5.

20. Procedure according to claim 18 which is characterized in that the peptide of step ii) with PTD/CPP-functionality comprises a TAT peptide.

21. Procedure according to claim 18 which is characterized in that the peptide of step ii) with PTD/CPP-functionality comprises a TAT-like peptide.

22. Procedure according to claim 18 which is characterized in that the peptide of step ii) has a decapeptide sequence GRKKKRRQRC.

23. Procedure according to claim 18 which is characterized in that the separation in step iii) is performed by ultrafiltration using a 10 kDa molecular weight cut-off membrane.

24. Procedure according to claim 18 which is characterized in that the polymer complexes are synthesized in isotonic glucose solution at pH 7.4.

25. Procedure according to claim 18 which is characterized in that the nucleic acid is added to the polymer complexes.

26. Procedure according to claim 18 which is characterized in that the nucleic acid is DNA and/or RNA.

27. Employment of a vector system according to claim 1 for pulmonary application.

28. Employment of a vector system according to claim 1 for inhalation.

29. Employment of a vector system according to claim 1 for the production of a medicament for the treatment of pulmonary diseases.

30. Employment of a vector system according to claim 1 for the production of a medicament for the treatment of e.g. pulmonary hypertony, mucoviscidosis, and lung tumors.
